(19) United States
(12) Patent Application Publication
(10) Pub. No.: US 2008/0082111 A1
(43) Pub. Date: Apr. 3, 2008

(54) Device and Method for the Evacuation of Matter Beneath the Skin

(76) Inventors: Richard Jaffee, Weston, CT (US); Peter H. Muller, Woodside, CA (US)

Correspondence Address: RUBENSTEIN & GLAZER, LLC, ONE MORNINGSIDE DRIVE, NORTH, BUILDING A, WESTPORT, CT, 06860, US

(21) Appl. No.: 11/745,378

(22) Filed: May 7, 2007

Related U.S. Application Data
(60) Provisional application No. 60/746,702, filed on May 8, 2006.

(51) Int. Cl. A61B 17/50

(52) U.S. Cl. 606/131

(57) ABSTRACT

A device and method used for the evacuation of matter beneath an area of skin comprising a squeezing interface comprising approximately concentric squeezing surfaces which apply pressure around the area of skin to force said matter from beneath the skin.

This application claims benefit under 35 U.S.C. 119(e) to Provisional Application No. 60/746,702 filed May 8, 2007.

### BACKGROUND OF THE INVENTION

1. Field of the Invention

This invention relates to a device and the method of using it for the evacuation of papules, pustules, vesicles; dermatological afflictions commonly referred to as pimples, zits, blackheads, whiteheads; as well as to other similar dermatological conditions that result in a collection of puss, blood, other fluid, bacteria, dirt, dead cells or dried or solid matter underneath the skin.

2. Description of Related Art

The underlying causes of these dermatological conditions are hard to treat and difficult to prevent. The visible, palpable manifestation of pimples, blackheads and other unsightly skin blemishes are often a source of personal discomfiture and even embarrassment. Accordingly, a person so afflicted often may attempt removal of the outward, visible manifestation by expelling their contents and reducing, at least temporarily, the associated swelling, even at the risk of pain and further infection.

While this invention relates to the evacuation and removal of many different forms of matter beneath the skin and other dermatological manifestations, as set forth above, merely for the sake of brevity and convenience, the description that follows will describe the evacuation and removal of pimples and blackheads.

A common, simple method of removing a pimple involves squeezing using fingers or even fingernails, until the pimple pops and the contents can be expelled. The pimple may also be evacuated by squeezing the pimple using some other readily available device, such as tweezers. Another common method involves lancing the pimple to facilitate the expulsion of the internal contents, often performed in combination with some subsequent squeezing to fully expel the contents.

There are many problems associated with squeezing a pimple between fingers and/or fingernails to forcibly expel its contents. For example, fingers are large in comparison to the area of the pimple so that visibility of the target may be impaired by the size and shape of the fingers. Squeezing in this manner becomes a clumsy, painfully inexact process. Sometimes the edges of fingernails are used for more precision and improved visibility, but fingernails are sharp and dirty and may cause lacerations and secondary infections. While the use of tweezers may avoid some of the above-mentioned disadvantages, tweezers have sharp edges, ridges and flat, longitudinal pressure surfaces which are not well suited to compressing pimples and the like. In this respect, tweezers and fingers cannot apply their squeezing force uniformly around the circumference of the pimple. Worse still, the flat gripping surface of the tweezers' face tends to pull the skin on and around the pimple, causing the already-taught, swollen skin to tear or to be cut by the tweezers' edge.

### SUMMARY OF THE INVENTION

The present invention provides a device and method for the removal of matter beneath the skin comprising:

A squeezing interface adapted to provide a more uniform squeezing force around the circumference of the pimple;

A squeezing interface which is adapted to slide across the surface of the skin while applying squeezing forces inwardly and, optionally, upwardly around the circumference of the pimple, reducing cutting and pulling forces on the skin;

A more easily and uniformly directable squeezing force to expel the contents of the pimple, preferably up and out of the top of the pimple head;

A device which is adapted to fit easily and securely between two fingers and which provides the user with greater control and precision in applying the necessary squeezing force; and

A device which provides a visually unobstructed squeezing interface.

The squeezing interface of the present invention comprises two or more opposing squeezing members comprising concave or convex (curved) squeezing faces between which the pimple may be placed. When the squeezing members are deployed by the user, the squeezing faces are brought together toward a central axis to contact the pimple and apply pressure at approximately symmetrical areas around the circumference of the pimple.

When the device's squeezing members are deployed into a closed or partially closed position, the opposing squeezing faces comprise partial or contiguous boundaries of an approximately round or elliptical lumen. The shape and the size of the lumen may be varied across devices of the present invention so that the user may select a preferred device according to the size and shape of the pimple to be treated. Any single device may treat a range of pimple sizes as the squeezing faces may contact the walls of the pimple through a range of deployed positions, before the squeezing members are fully deployed.

Optionally, the opposing squeezing faces may be angled, concentrically, from a lower, inward portion of the lumen, to an upper, outward portion of the lumen to provide an inward and upward vector of force as the squeezing faces come together, providing a scooping effect.

With the squeezing members in a fully open position, the device is placed against the surface of the skin with the squeezing faces loosely surrounding the target pimple. As the user deploys the squeezing members inward, the opposing concentric squeezing faces apply an inward, and optionally upward, pressure, more or less evenly around the circumference of the target pimple. The user applies a steady pressure to bring the squeezing members together, thereby increasing the pressure against and within the pimple. If the squeezing faces are angled, the force applied against the walls of the pimple is vectored upward and inward as the squeezing faces come together and consequently the pressure produced within the pimple is selectively directed against the upper inside portion of the wall of the pimple and the contents can be expelled out of the head of the pimple.

### BRIEF DESCRIPTION OF THE DRAWINGS

FIG. 1A shows a perspective view of a first preferred embodiment of the device of the present invention. FIGS. 1B and 1C show a front facing view of the same embodiment in an open and a closed configuration, respectively. FIGS. 1D and 1E show a profile view of the same embodiment applied to a skin surface in an open and closed configuration, respectively. FIGS. 1F and 1G show a top perspective view of the same embodiment in an open configuration and FIGS. 1H and 1I show a top perspective view of the embodiment in a closed configuration.

FIGS. 2A and 2B show perspective views of a second preferred embodiment in an open and closed configuration, respectively. FIGS. 2C and 2D show a front face of the same device in an open and closed position; respectively. FIG. 2E shows a profile view of the same device. FIGS. 2F and 2G show a top view of the same device in an open position and FIGS. 2H and 2I show a top view of the same device in a closed position.

FIGS. 3A and 3B show a top view and a side view, respectively, of a third preferred embodiment of the present invention in an open configuration and FIGS. 3C and 3D show a top view and a side view, respectively, of the same device in a closed configuration.

FIGS. 4A and 4B show a top view and a side view, respectively, of a fourth preferred embodiment of the present invention in an open configuration and FIGS. 4C and 4D show a top view and a side view, respectively, of the same device in a closed configuration.

FIGS. 5A and 5B show a top view and a side view, respectively, of a fifth preferred embodiment in an open configuration with FIG. 5C providing an expanded, partial detail view from FIG. 5B. FIGS. 5D and 5E show a top view and a side view, respectively, of the same device in a closed configuration.

FIGS. 6A and 6B show a top view and a side view, respectively, of a sixth preferred embodiment in an open configuration with FIG. 6C providing an expanded, partial detail view from FIG. 6B. FIGS. 6D and 6E show a top view and a side view, respectively, of the same device in a closed configuration.

### DETAILED DESCRIPTION OF PREFERRED EMBODIMENTS

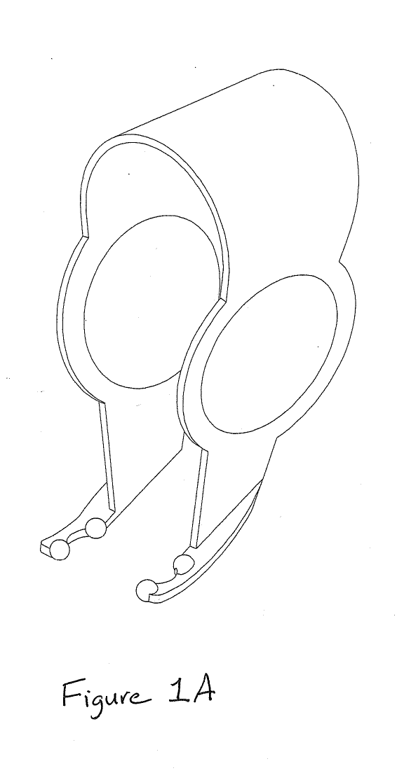

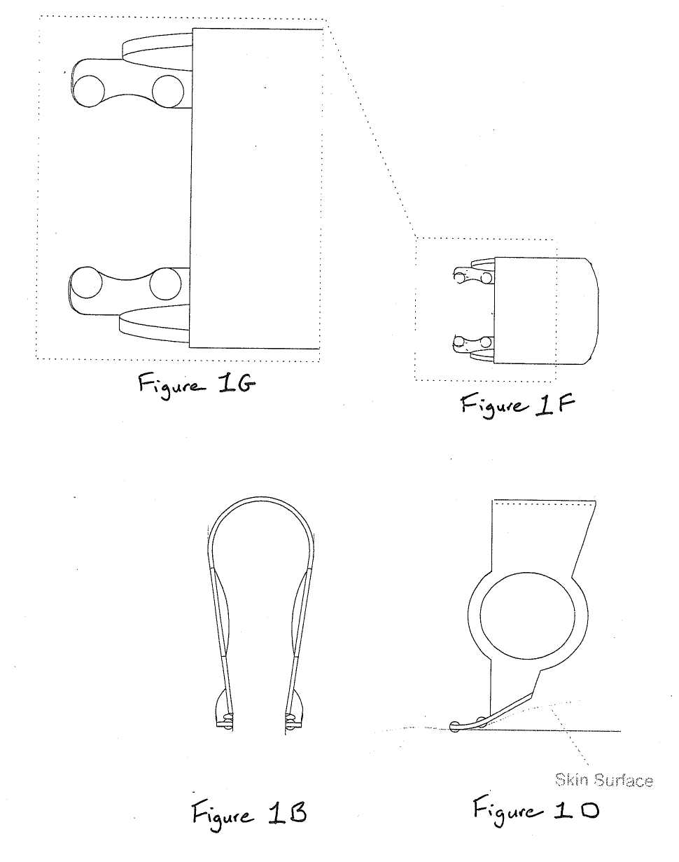

A preferred embodiment of the present invention, as shown in FIGS. 1A through 1E, comprises a U-shaped device with two lateral arms, a tension bridge connecting the lateral arms and a distal squeezing interface extending from the open end of the arms. The squeezing members in this embodiment comprise two opposing squeezing arms, each of which extends in a forward direction off one of the two lateral arms and two squeezing faces, each comprising a concave recess along an interior side of the opposing squeezing arms.

A section on each of the lateral arms is of a suitable width and length to securely accommodate a user's fingers or knuckles. Optionally, the two lateral arms may comprise approximately thumb-sized round and, optionally concave opposing grasping sections to securely accommodate a user's fingers. These grasping sections may comprise a material which will improve user comfort and grip, such as a textured plastic or soft rubber, or any other material which will be familiar to one skilled in the art.

The device may be grasped using one finger along each arm, preferably at the finger-holds. The device may be closed by squeezing against the outward tension provided by the tension bridge until the opposing squeezing arms are brought together. The lateral arms of this embodiment are bowed outward to provide an optimal grasping and squeezing configuration. When the squeezing arms are pushed together into a closed position, the two opposing squeezing faces form the walls of an approximately elliptically-shaped lumen.

The squeezing arms may be affixed along the entire portion of the bottom of the lateral arms and may extend off the lateral arms at an angle from approximately 0 to approximately 160 degrees, preferably at about 45 to 65 degrees, relative to the front edge of the lateral arm. The bottom portion of the lateral arm also may be angled or sloped from the front edge of the lateral arm to the back edge of the later arm. The angle or angles provided by the juncture of the squeezing arm to the lateral arm and the slope of the bottom of the lateral arm all may be selected to provide optimum visibility of the squeezing interface, to provide the optimum contact between skin and squeezing interface and to minimize contact with the body by portions of the device other than the squeezing interface.

A portion of the squeezing arms may optionally be curved or bowed downward, toward the skin, preferably such that the apex of the curve coincides approximately with the center of the squeezing face. The degree of the curvature across the bowed portion may be variably selected to optimally position the center of the squeezing faces at the deepest, centermost base of the pimple.

The squeezing faces may be angled from approximately 0 degrees to approximately 85 degrees sloping upward and outward, from the inside bottom portion of the squeezing face to the outside top portion of the squeezing face. In this angled configuration, the bottom circumference of the lumen is smaller than the circumference of the top portion of the lumen such that as the squeezing faces come together, the forces against the circumference of the pimple walls are vectored upward. In this way, the contents of the pimple may be expelled preferably out of the top of the pimple. The device in this embodiment is designed to further allow the user to lift the squeezing interface away from the surface of the skin in a closed or partially closed position to use the angled squeezing faces to force or “squeegee” the contents of the pimple from the bottom or base of the pimple, out of the top of the pimple.

The squeezing interface of the present invention optionally may also comprise a pair or pairs of spherical or partially-spherical protruding skin guides located on the squeezing arms approximately adjacent to one or both ends of the squeezing faces. As the opposing squeezing arms come together, the bottom rounded portion of the skin guides pass along the surface of the skin, and the protuberance guides or rolls the skin ahead, above and below the squeezing face. This action is designed to assist in the collection of the body of the pimple into the lumen and to further prevent the pulling and cutting associated with conventional pimple popping methods and devices.

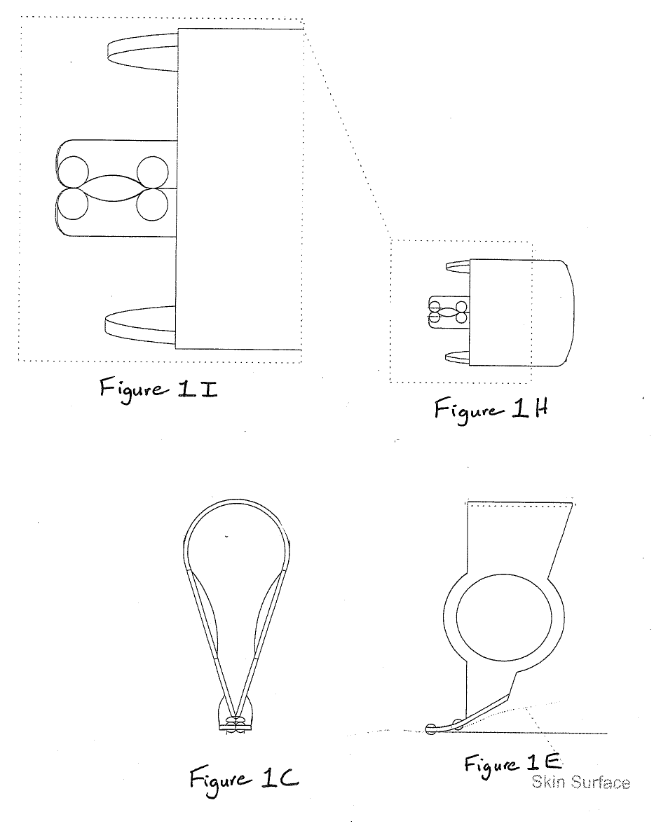

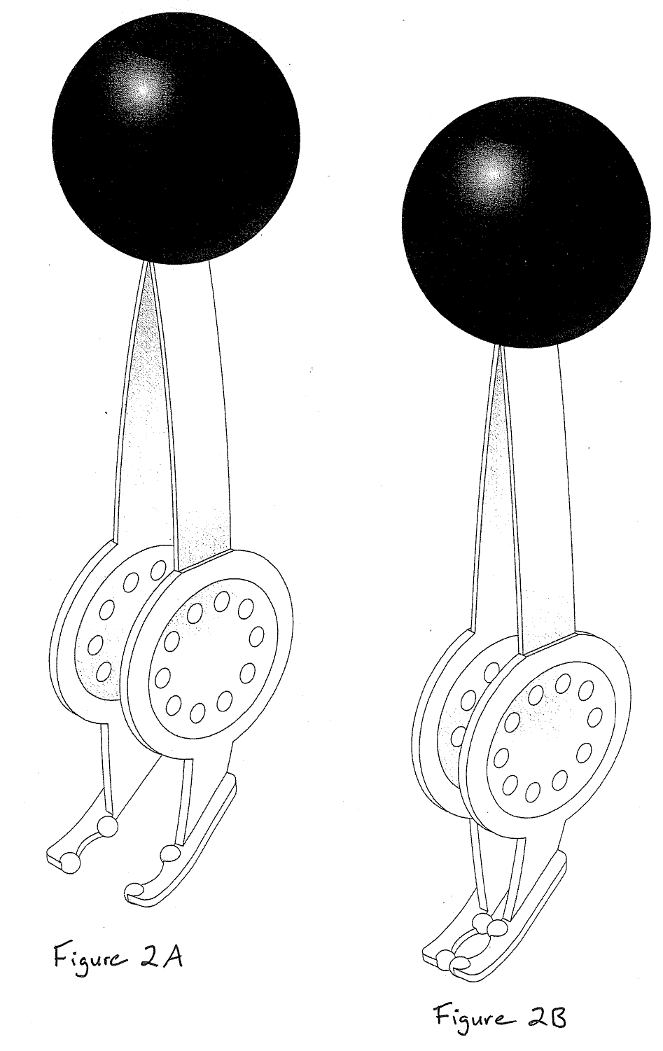

A second preferred embodiment of the present invention, as shown in FIGS. 2A through 2I, comprises an ergonomic knob adapted to allow the user to more securely grasp the device and to provide additional comfort precision during use. The ergonomic knob may be adapted to fit into the user's palm. In this preferred embodiment, an ergonomic rounded bulb-like handle is affixed at the top portion of the device, at or around the juncture of the two opposing lateral arms. The user of this embodiment may grasp the ergonomic knob in the crux of the palm for additional comfort and stability while using fingers to grasp and squeeze the lateral arms, preferably at finger holds provided thereon. Optionally, the ergonomic knob may have a flattened surface, preferably at the top, which provides the knob with the additional ability to function as a stand such that the device may be rested on a surface to avoid having the squeezing faces coming into contact with any contaminating surfaces.

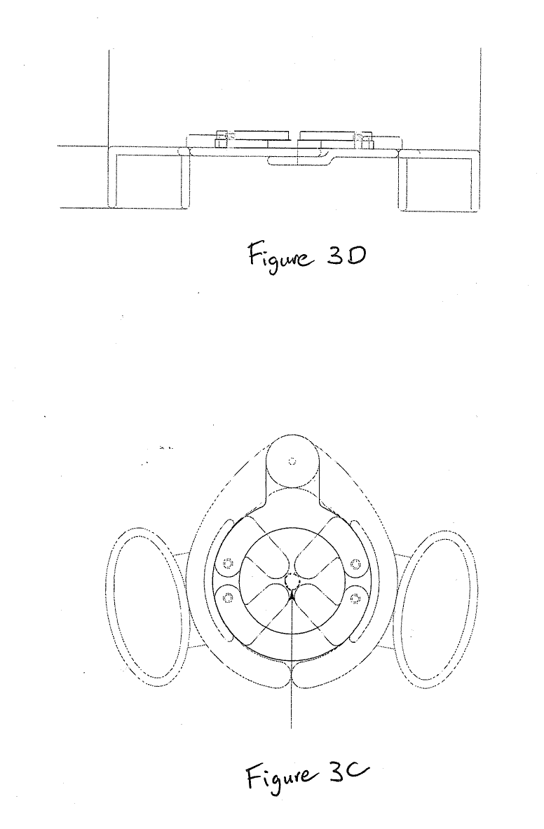

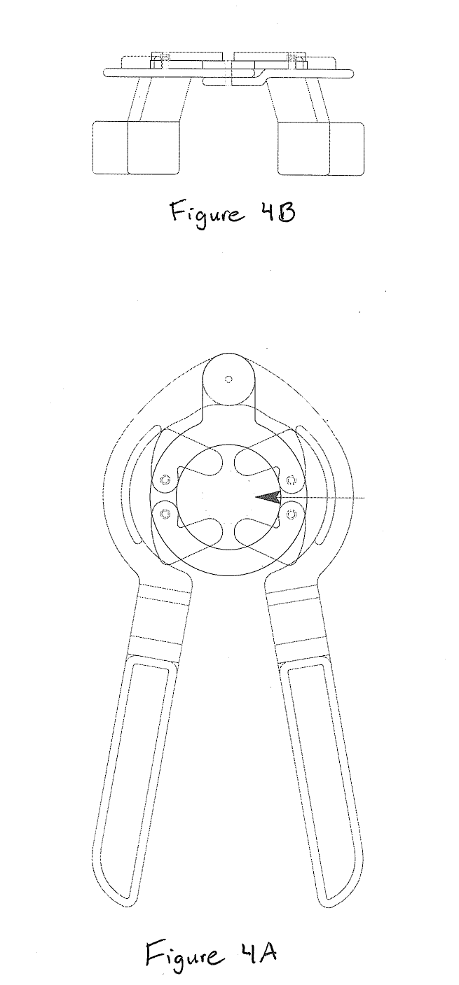

A third preferred embodiment of the present invention, as shown in FIGS. 4A through 4E, comprises an inner mounting ring on which are mounted two or more squeezing members and a deployment interface which transfers forces applied by the user to the squeezing members and ultimately to the pimple. In this embodiment, the squeezing members are moveably affixed to the mounting ring such that the squeezing members may be moved concentrically inward to apply pressure to a pimple via the opposing squeezing faces. Means for affixing the squeezing members to the mounting ring may comprise, among other well-known means, a pivot or a slot in combination with a spring means for returning the squeezing members to an open, pre-deployment position.

The deployment interface comprises an outer, approximately horseshoe-shaped open ring comprising a guide surface for contacting the squeezing members and gripping means for holding the device and actuating the squeezing members. In this embodiment, a handle extends off each bottom end of the open ring such that they the handles may be gripped and brought together by the user, thereby partially closing the open ring and causing the guiding surfaces to contact the squeezing members, pushing them into a closed or partially closed position. When the device is fully deployed, the handles are brought together along their inner faces.

The open ring may be hinged at an apex to allow the ring to partially close, with the hinge being provided in combination with spring means for providing squeezing resistance and a returning force to urge the ring back into an open position. Alternatively, the open ring may comprise an elastic portion which allows the ring to partially close upon application of pressure by the user and to re-open once the pressure is released.

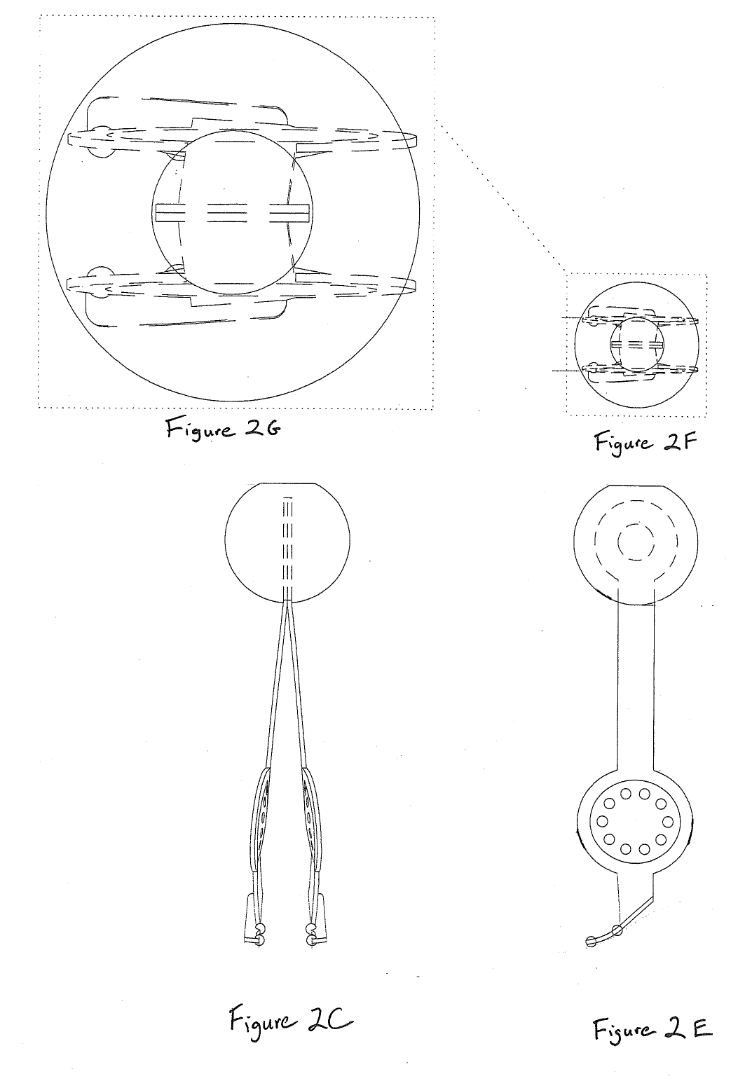

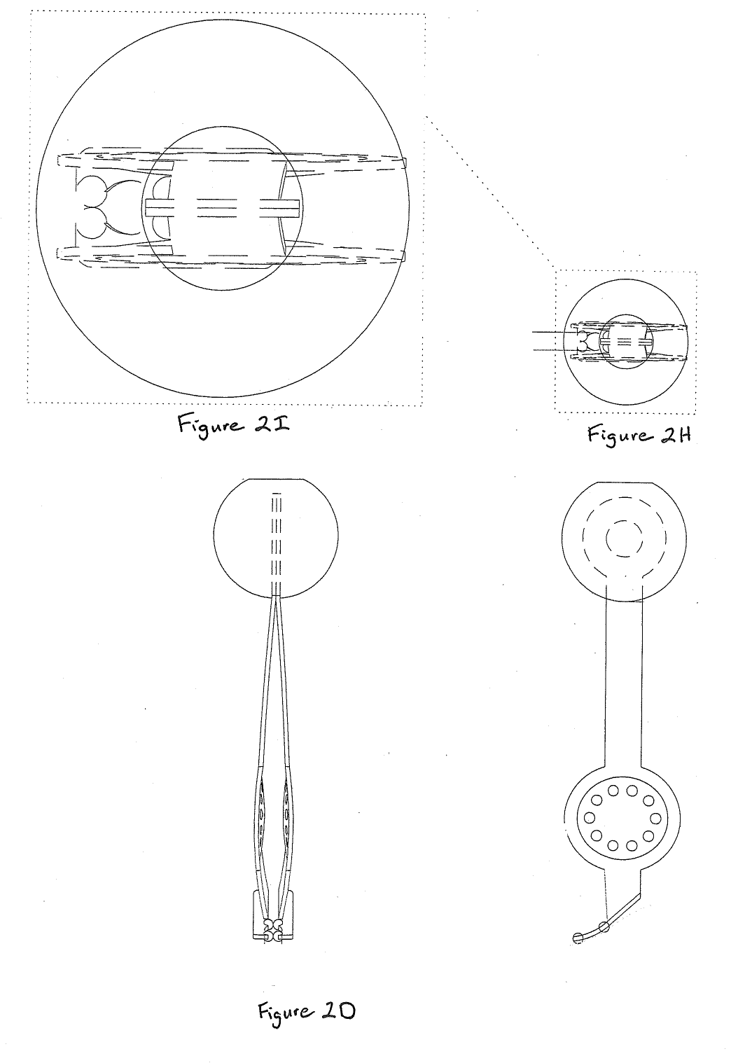

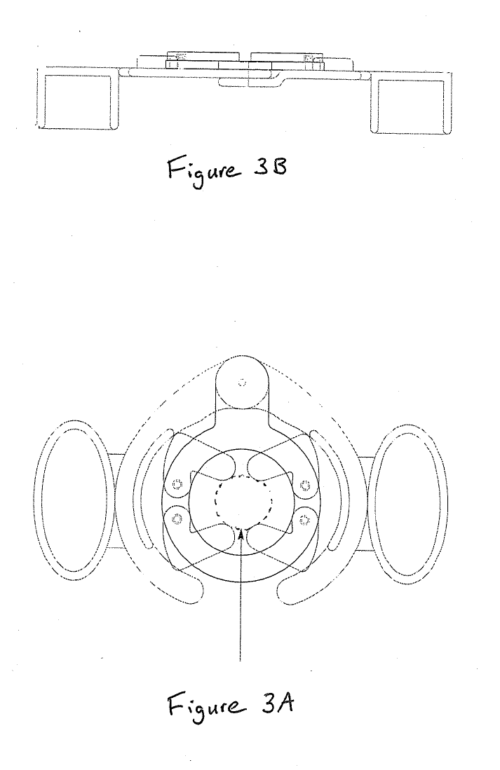

In a fourth preferred embodiment, as shown in FIGS. 3A through 3C, the handles extending off a bottom portion of the open ring are replaced by gripping means located on opposite sides of the open ring. Preferably, the gripping means are adapted to fit two of the user's fingers such that the familiar squeezing motion of the user's fingers will be applied via the device more uniformly and safely around the circumference of the pimple.

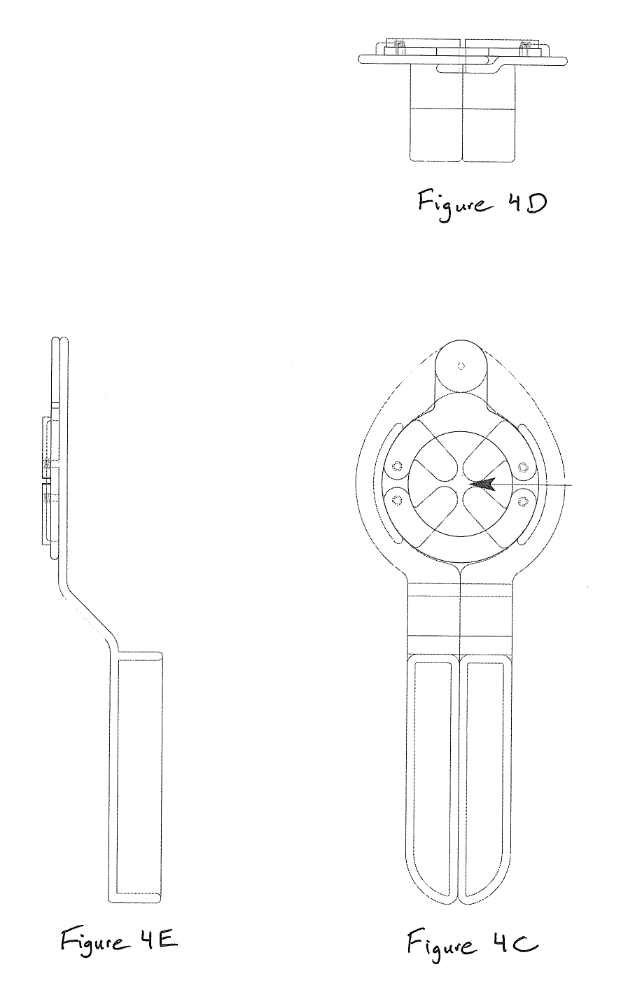

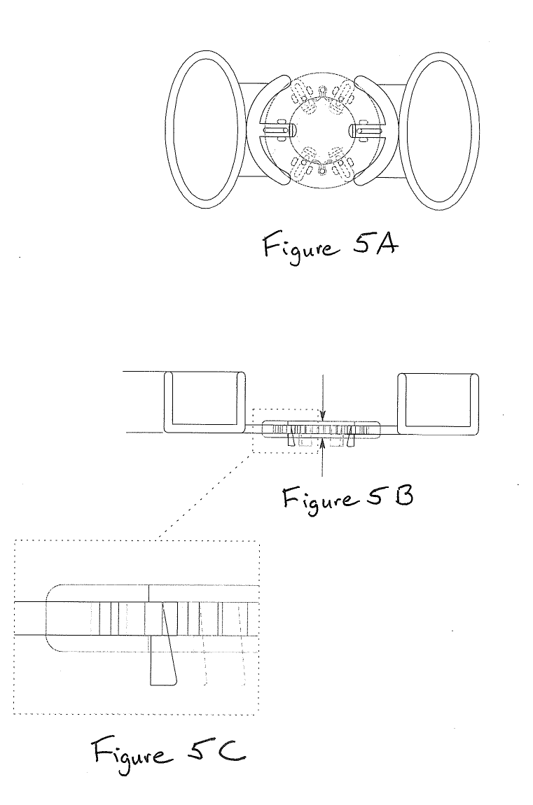

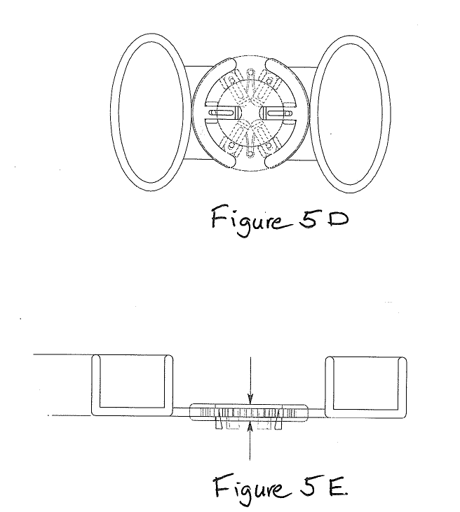

A fifth preferred embodiment, as shown in FIGS. 5A through 5E, comprises multiple squeezing members, with some squeezing members affixed to an inner mounting ring, and optionally with some squeezing member affixed directly to a deployment interface.

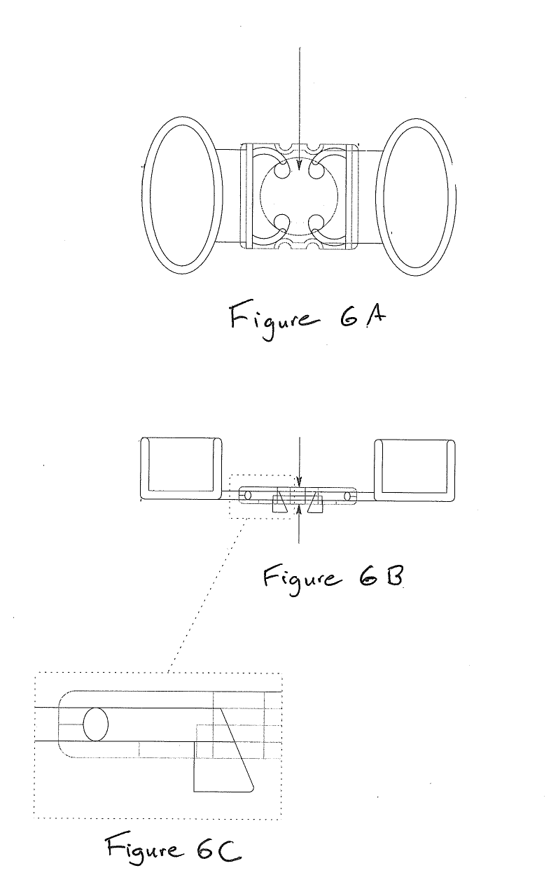

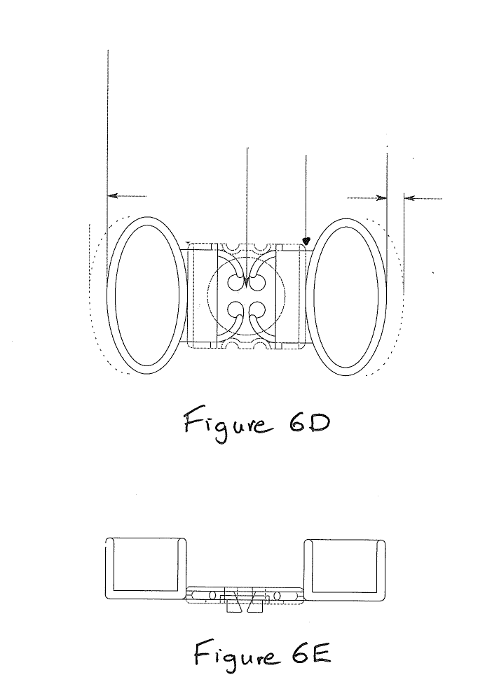

A sixth preferred embodiment, as shown in FIGS. 6A through 6E, comprises a deployment interface connected directly to squeezing members. Two or more squeezing members are connected directly to one of two opposing gripping means, via squeezing arms, to form a squeezing assembly. The opposing squeezing assemblies are both affixed to a central bracketing means which allows the user to slide the opposing squeezing assemblies together in a guided, controlled manner. As the user deploys the squeezing members inward, through the travel area allowed by the bracketing means, pairs of guiding ridges incorporated into the bracketing means deflect the squeezing arms of a squeezing assembly toward each other, causing the squeezing faces to come together at a concentric point.

The device in these embodiments may also be used as a more traditional comedone extractor. When the squeezing interface is fully closed, the blackhead may be encircled by the lumen provided by the squeezing faces and the user may thereby apply appropriate pressure to the skin surrounding the blackhead to force the trapped contents of the blackhead out and to the surface of the skin.

The materials used in the construction of any of the components of the devices of the present invention can be chosen from among all metals, rubbers, plastics, composite materials and any combination thereof, the various advantages and disadvantages of which will be obvious to one of ordinary skill in the art.

The foregoing descriptions of specific embodiments thereof are provided by way of examples of the present invention. It will be apparent to one skilled in the art that various changes, combinations and modifications thereof can be made without departing from the spirit and scope of the present invention.

We claim:

1. A device for the evacuation of matter beneath an area of skin comprising a squeezing interface comprising squeezing members comprising approximately concentric squeezing faces which deploy in an inward direction to apply pressure around said area of skin to force said matter from beneath the skin.

2. A method of evacuating matter from beneath an area of skin comprising the steps of placing the device of claim 1 in an open configuration loosely around said area of skin to be evacuated; squeezing lateral arms of said device to bring squeezing faces of said device into contact with said skin; and continuing to squeeze said arms until said matter is expressed out of said skin.
